(19) United States
(12) Patent Application Publication
(10) Pub. No.: US 2025/0009548 A1
(43) Pub. Date: Jan. 9, 2025

(54) SYSTEM FOR COLLECTING WASTE OF AN OSTOMY APPLIANCE

(71) Applicant: Mark Stensrude, Prior Lake, MN (US)

(72) Inventor: Mark Stensrude, Prior Lake, MN (US)

(21) Appl. No.: 18/739,129

(22) Filed: Jun. 10, 2024

Related U.S. Application Data
(60) Provisional application No. 63/507,008, filed on Jun. 8, 2023.
(60) Provisional application No. 63/658,029, filed on Jun. 10, 2024.

(51) Int. Cl. A61F 5/44

(52) U.S. Cl. CPC A61F 5/4404

(57) ABSTRACT

A system for collecting waste of an ostomy device. An input connection and an output connection are on opposite ends of a tube, and are respectively couplable to an ostomy device and a reservoir. Tubing reinforcement accessories support the system tubing at the connection points and inhibit kinks from forming in the tubing during use.

### CROSS REFERENCE TO RELATED APPLICATIONS

The present application claims the benefit of U.S. Provisional Application No. 63/507,008, filed Jun. 8, 2023, and U.S. Provisional Application No. 63/658,029, filed Jun. 10, 2024, which applications are incorporated herein by reference in their entirety.

### FIELD OF INVENTION

The present invention relates to a collection system of a wearer of an ostomy appliance that expels waste from a stoma.

### BACKGROUND

Certain medical procedures and conditions require the temporary or perpetual use of an ostomy appliance. Ostomy appliances are known in the art. These appliances are movably attached to a wearer and aid the wearer in expelling various types of bodily waste products via a stoma. Current ostomy collection devices can be worn against the wearers body or nearby the wearer and connected to the ostomy appliance via tubing. Wearers must empty or change the collection devices frequently, when they become full, as well as regularly clean them to prevent potential infections.

Urostomy users have medical conditions or procedures by which ostomy appliances function as their bladder, collecting expelled urine from the wearer. Urostomy patients are advised to heavily hydrate because a constant excretion of fluid through such an ostomy appliance aids in preventing infection and promoting general wellness. Because of the limited capacity of current ostomy collection devices and such urostomy users' heavy hydration, these wearers must empty or change their devices frequently. This is not only an inconvenience but also a hindrance in their day-to-day life. In a single night of sleep, such users would be forced to wake up and leave bed to empty their devices multiple times. Users would be similarly affected during the daytime and workday activities.

If a user fails to frequently empty their collection device, they risk spillage of waste from the device. To cope with the limited capacity of their ostomy collection systems, users may choose to limit their fluid intake while in a public setting. This not only inhibits their freedom to enjoy their time but also could be a risk to their health if they have been advised to increase their fluid intake to prevent infection at the site of their stoma.

Capacity alone is not the only problem wearers face for nighttime or other scenarios that call for extended time between emptying or changing their ostomy collection device. The few higher capacity options available utilize thin-walled polymeric bags as the collection option. These bag systems are unwieldly for wearers to deal with, particularly when used at their highest capacity. This can lead to spillage. These bag-like systems pose more issues for wearers who have additional medical conditions that may impede their mobility, because the flexible nature of the bag makes them difficult to grasp or transport to a location where a user would empty it (i.e., a bathroom). Additionally, the flexible nature of these reservoirs makes them difficult to completely empty of waste or fluids, which makes cleaning the system properly cumbersome. Alternative collection devices that utilize a reservoir with rigid walls have a smaller capacity than what would be required to allow the user an uninterrupted night's sleep.

Further, current ostomy device collection systems comprise flexible, thin-walled tubing that easily kinks, preventing the expelling of waste and posing a risk of spillage or even infection. The points of connection between the tubing and the stoma and collection reservoir are particularly vulnerable to this occurrence. Certain ostomy devices also utilize relatively short tubing, which can be problematic for extended use scenarios like sleeping or the workday, as users can be prevented from making small movements like rolling over or reaching across a desk.

The available systems that utilize a rigid container comprise a cap that is affixed to the tubing. After a few uses these systems begin to leak because of the flimsy nature of the tubing and the inadequate strength of the connection between the tubing and the cap. This leads to frustrating leaks that limit the user's ability to rely on their collection device.

There is a demonstrated need by ostomy appliance users for an ostomy collection system that has a greater capacity to enable users more time in between emptying their devices, while still being accessible to users who have limited mobility, as well as tubing that is shielded from kinks that cause spillage or infection. Users of ostomy appliances need a device that gives them the confidence to live their lives to the fullest and freedom to enjoy activities and full nights' sleep without constantly worrying about whether their ostomy appliance will leak or overflow.

### SUMMARY

The problems described above are in large measure solved by the system described in this application. According to embodiments, a system for collecting waste of an ostomy device includes an ostomy input connection, tubing, an output connection, tubing reinforcement accessories, and a reservoir. The input and output connections are couplable to an ostomy device and the reservoir, respectively. The tubing reinforcement accessories support the system tubing at the connection points to inhibit kinks from forming in the tubing during use of the system. With this system, users will not be inhibited by the capacity and construction of their ostomy collection devices and have the confidence and freedom to live their life uninterrupted by frequent trips to empty their collection devices.

### BRIEF DESCRIPTION OF THE DRAWINGS

The invention may be more completely understood in consideration of the following detailed description of embodiments of the invention in connection with the accompanying drawings, in which:

FIG. 1 is a perspective view of a system for collecting waste of an ostomy appliance;

FIG. 2 is a perspective view of a system for collecting waste of an ostomy appliance, having a carrier;

FIG. 3 is a partial perspective view of a reservoir of a system for collecting waste of an ostomy appliance;

FIG. 4 is a partial perspective view of a system for collecting waste of an ostomy appliance, having a reinforcement accessory at a connection point;

FIG. 5 is a perspective view of a reinforced connection valve for a system for collecting waste of an ostomy appliance;

FIG. 6 is a close up, perspective view of the connection valve of FIG. 5;

FIG. 7 is a close up, partial perspective view of a connection valve of a system for collecting waste of an ostomy appliance, having a reinforced attachment to some tubing;

FIG. 8 is a perspective view of an example of related art; and

FIG. 9 is a perspective view of an example of related art.

FIG. 10 is a perspective view of a home use cannister for an alternative embodiment of a system for collecting waste of an ostomy appliance;

FIG. 11 is an exploded view of the cap/tube connector of the system for collecting waste of an ostomy appliance of FIG. 11.

FIG. 12 is similar to FIG. 11, but depicting the cap/tube connector juxtaposed with the home use cannister in an exploded view;

FIG. 13 is similar to FIG. 12, but depicting the cap/tube connector coupled together in an exploded view in relation to the cannister of the system for collecting waste of FIG. 10;

FIG. 14 is a perspective view of travel pack cannister for an embodiment of a system for collecting waste of an ostomy appliance, including a partial enlarged and exploded view of the tube connector, tube support and tube fitting of the travel pack cannister;

FIG. 15A is a perspective view of the travel pack cannister partially received within a backpack; and

FIG. 15B is similar to 15A, but with the travel pack cannister indicated in phantom lines, fully received within the travel pack cannister.

While embodiments are amenable to various modifications and alternative forms, specifics thereof have been shown by way of example in the drawings and will be described in detail. It should be understood that the intention is not to limit the invention to the particular embodiments described. The intention is to cover all modifications, equivalents, and alternatives falling within the spirit and scope of the invention as defined by the appended claims.

### DETAILED DESCRIPTION

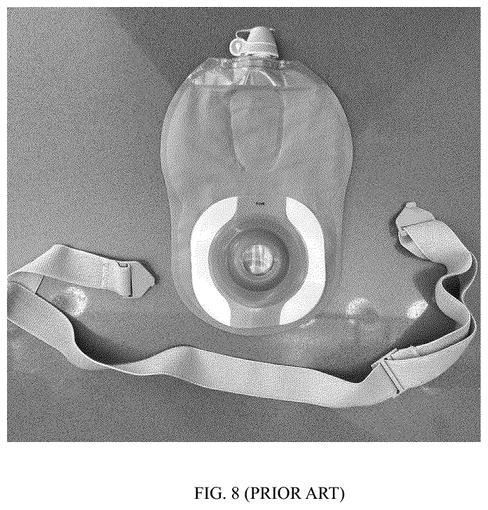

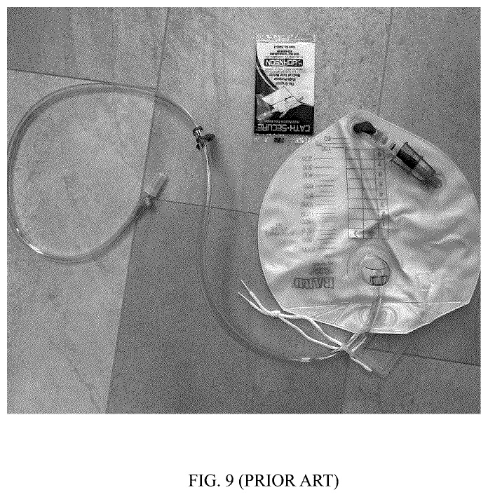

Current ostomy collection systems inhibit wearers comfort and mobility because of the systems' construction and capacity. FIG. 8 demonstrates a known embodiment of ostomy collection systems, which utilizes a small bag-like reservoir that is worn against the user's body. The capacity of this particular system is limited to only one-third that of a human bladder, which severely limits wearers freedom in their day-to-day activities. FIG. 9 demonstrates another such embodiment, intended as a nighttime specific solution, where the limited length of tubing inhibits movement of users, such as turning or rolling over while laying down, and the flimsy construction of the reservoir can be difficult to empty. Both these known ostomy collection systems are also cumbersome to clean because of flexibility of the reservoirs.

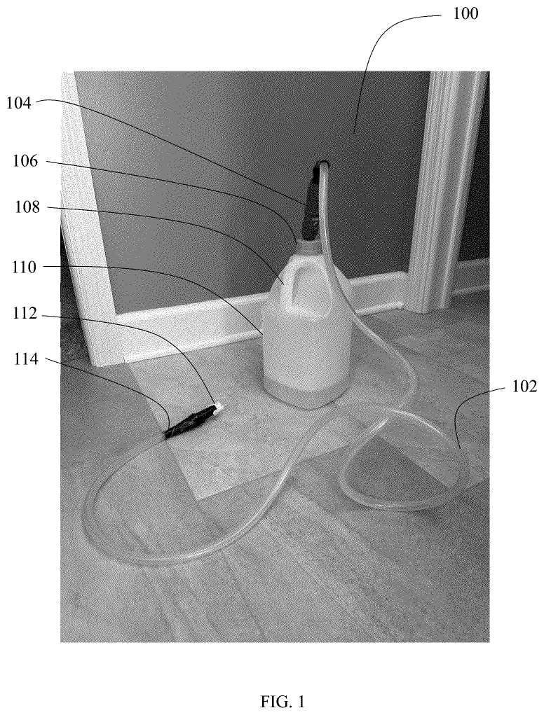

According to an embodiment, FIG. 1 shows a system 100 for collecting waste from ostomy appliances, include a reservoir 110, a cap 106, tubing 102, and an input connection 112. The tubing 102 is reinforced at its connection points with the reservoir 110 and an ostomy appliance (not pictured) by a tubing reinforcement accessory 104 and bracing 114, respectively. In normal use, the reservoir 110 holds fluid that drains from the ostomy appliance, draining by flowing through the input connection 112, through the tubing 102, and into the reservoir 110 via the cap 106, when the cap 106 is affixed to the reservoir 110.

In the embodiment depicted in FIG. 1, users can empty the waste collected in the reservoir 110 by removing the cap 106. Users may also clean the system 100 in the same way. Because the reservoir is a rigid material, cleaning is a less cumbersome process because collected waste will not get caught in the folds or kinks that occur in other, flexible reservoirs. Once the user has performed the desired emptying or cleaning of the system 100, the user can replace the cap 106 and the system 100 is ready to collect waste from the ostomy appliance.

The tubing 102 is contemplated to have a material durometer high enough such that the tubing 102 is resistant to folding over itself or kinking. Further, the tubing 102 is contemplated to be around 72 inches in length, such that users can utilize the system 100 during sleep without being inhibited in movements like rolling over or turning on their side. The material of the tubing 102 is contemplated to be constructed from any material medically suitable for an ostomy collection device.

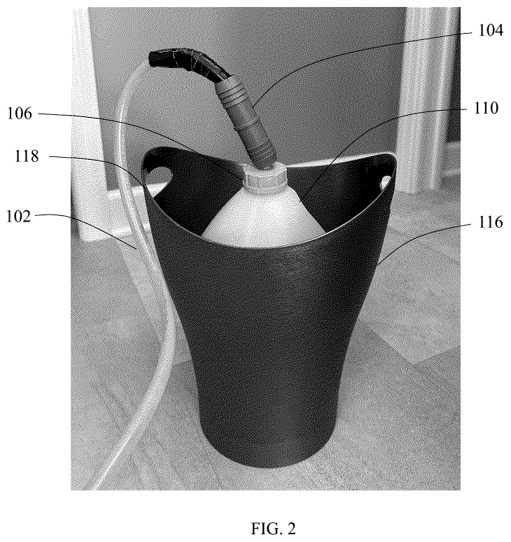

FIG. 2 shows the system 100 of FIG. 1 placed within a carrier 116. The carrier 116, as pictured, has a handle 118 on each side. This is by no means the only configuration of carrying aids contemplated and only serves as an example. The carrier 116 serves to improve mobility for users of ostomy appliances, by simplifying the transport of their collection devices. The carrier 116 may be constructed of various suitable materials, including the pictured rigid polymer materials. The carrier 116 may have multiple handles 118, or a singular handle 118, which may be constructed as part of the carrier 116, as shown, or as a movable attachment or any other similar configuration.

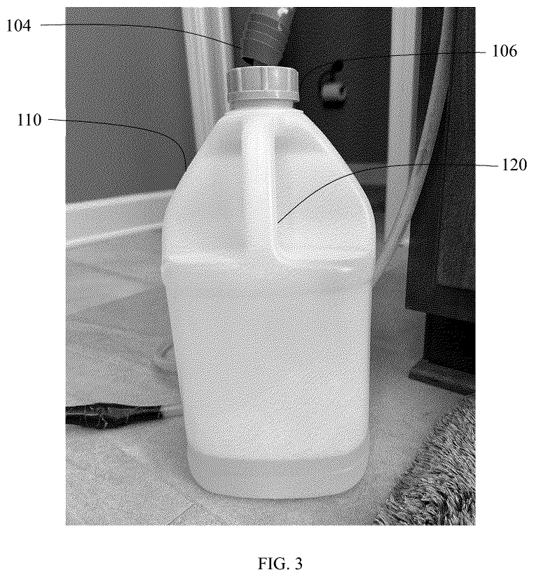

FIG. 3 demonstrates one contemplated embodiment of the reservoir 110. The reservoir 110 is constructed of a sturdy material, such as a rigid polymer or other such material that is compatible with the medical requirements of an ostomy collection device. The cap 106 is movably affixed to the reservoir 110, such that the user can remove the cap 106 to empty, clean, etc. the reservoir 110 and then replace the cap 106 for continued use of the system 100. The tubing 102 (not visible in FIG. 3) is affixed to the cap 106 in this particular embodiment, such that the tubing 102 drains through the part of the cap 106 that the tubing 102 is affixed to. The tubing 102 connection to the cap 106 is protected in this embodiment by the tubing reinforcement accessory 104, to inhibit kinks in the tubing 102 that can lead to spillage or even infection. The connection between the tubing 102 and the cap 106 is airtight to prevent leakage as fluids drain through the tubing 102 and into the reservoir 110.

In the embodiment shown in FIG. 3, the reservoir 110 has a handle 120. The functionality of the handle 120 is similar to that of the carrier 116 of FIG. 2, discussed supra. The reservoir 110 may include a handle 120. It is contemplated that the reservoir 110 may not have a handle 120, particularly in an embodiment that includes a carrier 116.

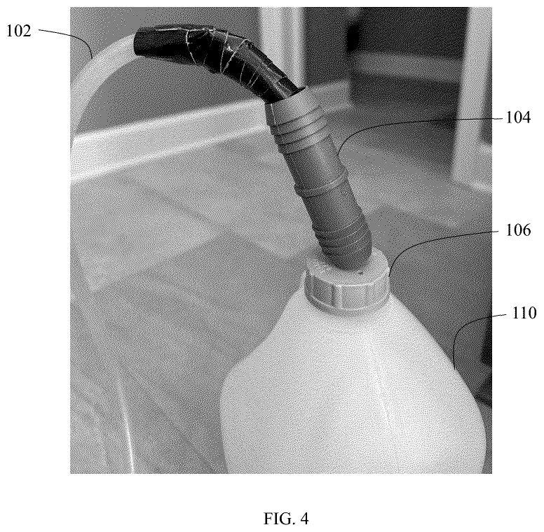

The connection between the tubing 102 and the cap 106 in an embodiment is demonstrated in FIG. 4. The tubing reinforcement accessory 104 supports the tubing 102 at its connection point to the cap 106. The tubing 102 may be connected to the cap 106 in any suitable method known or to be known in the art, such that the tubing 102 drains its' contents through the cap 106 into the reservoir 110.

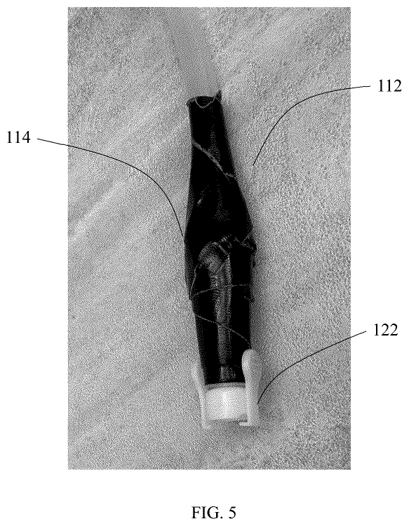

The input connection 112 connects the system 100 to the ostomy appliance (not pictured) worn by the user. FIG. 5 demonstrates one contemplation of the construction of the input connection 112. Bracing 114 protects the tubing 102 near the input valve 122. The bracing 114 inhibits kinks or folds in the tubing 102 near the input valve 122. This is important because back-ups in the expelled waste are particularly concerning this close to the user's ostomy appliance, as it would inhibit waste from being expelled by the stoma or cause spillage.

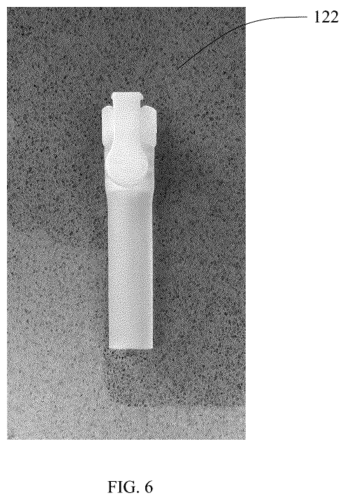

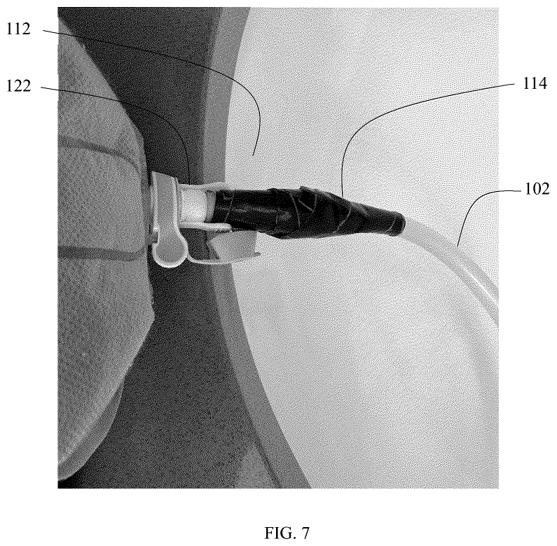

FIG. 6 is one contemplated construction of the input valve 122, depicted prior to connection to the tubing 102. The input valve 122 should be connected to the tubing 102 such that the connection between the input valve 122 and tubing 102 is completely airtight. This may be accomplished by a manufacturing process where the input valve 122 is molded directly to the tubing 102. This may also be accomplished by a system that includes accessories that enable the user to affix the tubing 102 and the input valve 122 with an airtight connection. Such accessories may include an adhesive that forms a rigid polyurethane polymer when activated or an adhesive tape.

In some embodiments, the input valve 122 is a universal connector. Such universal connectors enable the input valve 122 to be compatible with many available ostomy appliances.

In alternative embodiments, system 100 is modified for discrete public use. Such an alternative system (not shown) does not utilize a rigid container, instead comprises a flexible-walled container. The user may discreetly wear this embodiment by attaching the flexible-walled container to their waist or leg with a belt. The connections and tubing of the system are largely the same as that described in system 100, with the necessary adaptations for this type of use.

Users, instead of wearing this alternative collection system, may place the flexible walled container in a wearable carrying aid, like a backpack or a tote bag. Ostomy collection systems typically rely on gravity to drain waste from the ostomy appliances. In these proposed embodiments, gravity fed collection may be an option. However, in the systems where the flexible walled container is transported in a carrier like a backpack or a tote bag, relying on gravity may not be sufficient. In this instance, a low flow pump may be included in the system to aid with the flow of waste through the tubing into the flexible walled container.

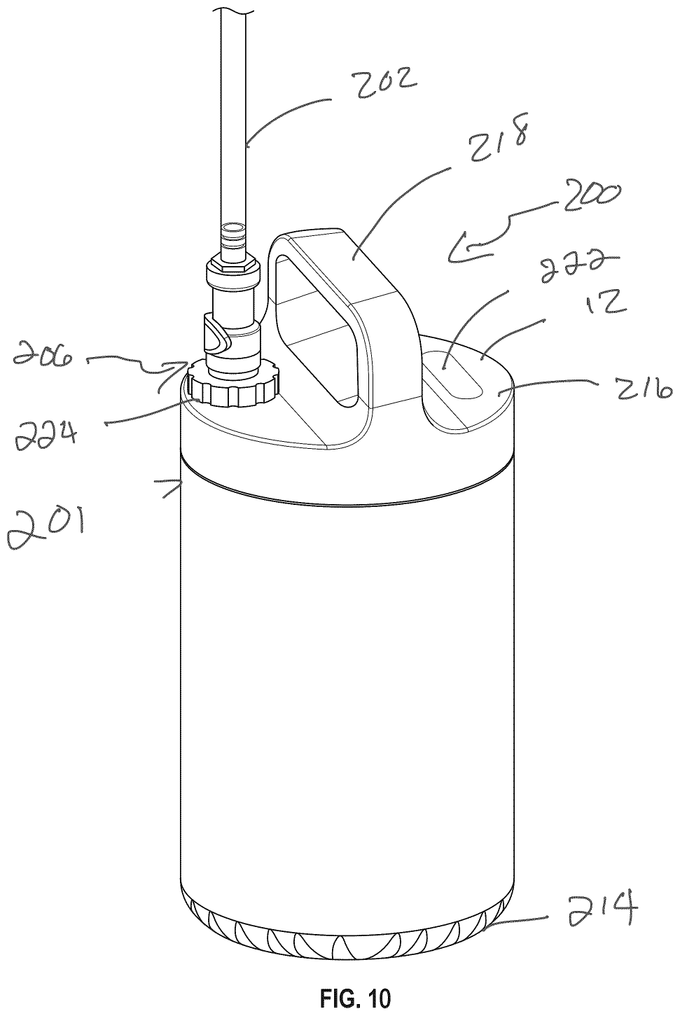

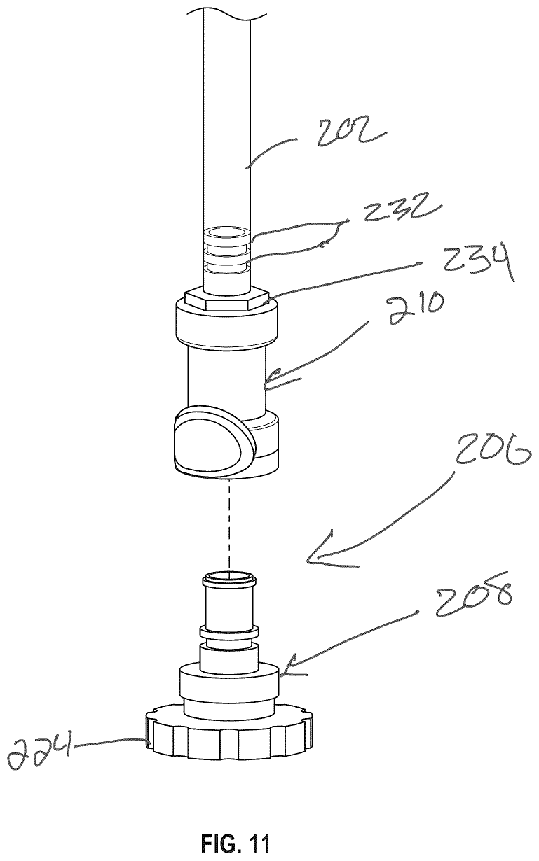

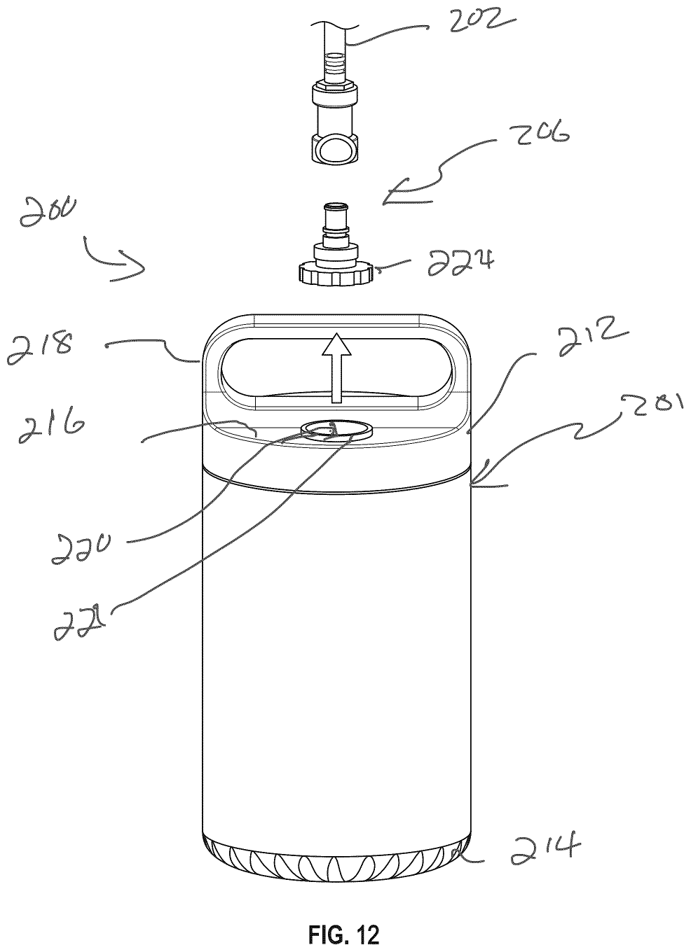

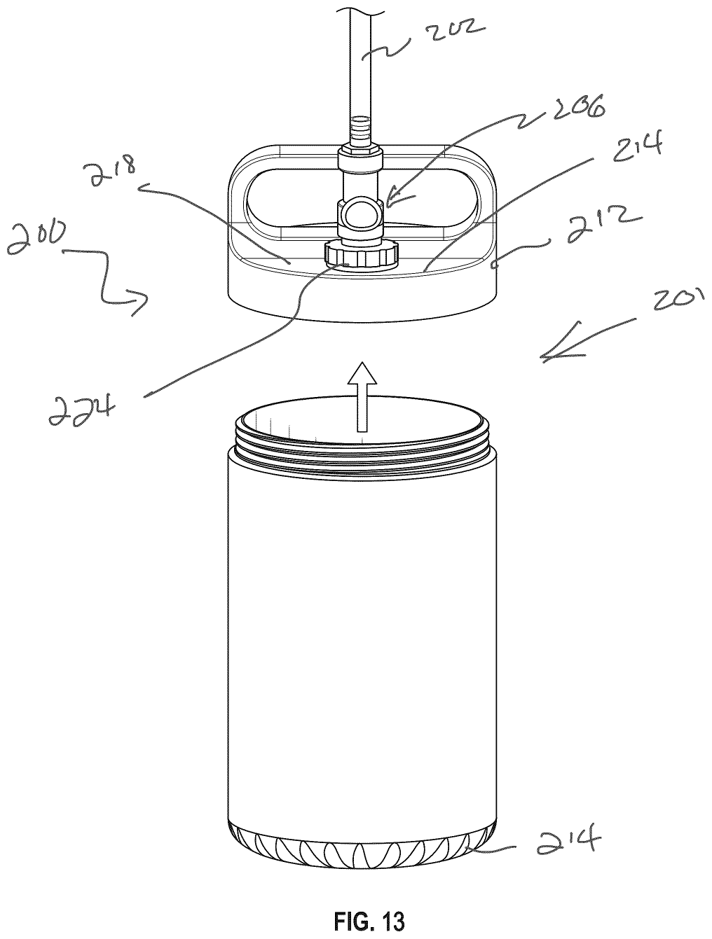

FIGS. 10-13 depict an alternative embodiment of a system 200 for collecting waste of an ostomy appliance in accordance with the invention. The system 200 for collecting waste from ostomy appliances, includes a reservoir 201, tubing 202, reservoir fitting 206 and an input connection (not shown).

The reservoir 201 includes a cannister 210 adapted for threadably receiving a cap 212. The cannister 210 can advantageously hold a gallon of fluid. The cannister 210 can include a resilient base 214, such as a rubber base. The cap 212 can include a top surface 216, handle 218, aperture 220, and viewing window 222. Aperture 220 includes an outwardly extending lip 221.

Fitting 206 includes cap 224 and tube connector 226. Cap 224 can be threadably or snappably connected to aperture lip 221. Tube connector 206 includes connector male portion 208 and female portion 210. The female portion 210 can be adapted for snappably being received by the male portion 208 in a fluid tight fit. Female portion 202 is further adapted for receiving the tube 202 in fluid tight fit, with fiction fit ridges 232 and tightening nut 236.

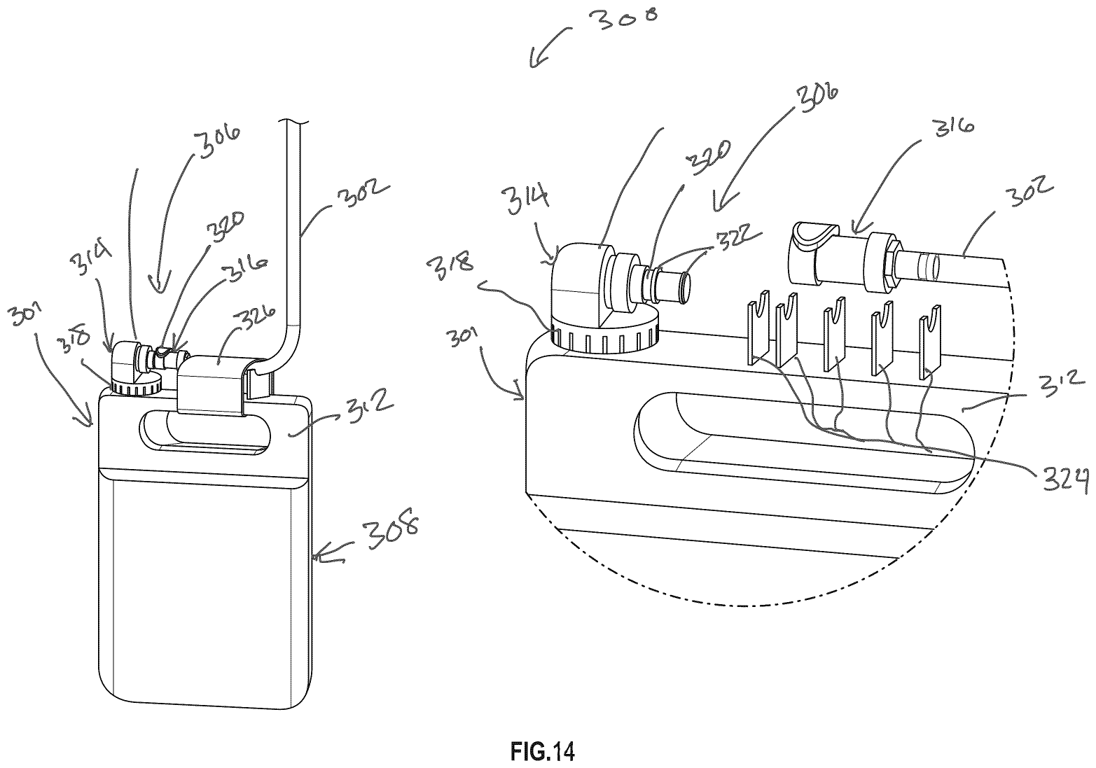

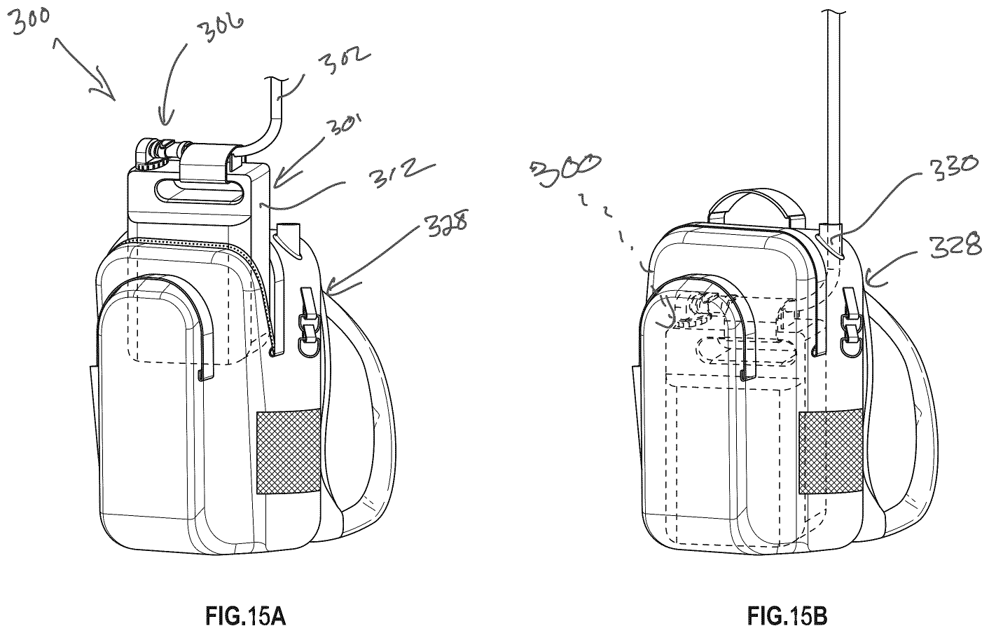

Referring to FIGS. 14 and 15A and 15B, a travel pack embodiment 300 of a system for collecting waste of an ostomy appliance includes a reservoir 301, tubing 302, fitting 306 and an input connection (not shown).

The reservoir 301 includes a cannister 308 having a port (not shown) adapted for threadably receiving fitting 306. The cannister 308 includes a port (not shown) adapted for detachable, fluid tight coupling with fitting 306. The cannister 308 further includes structure defining a hand graspable handle portion 312.

Fitting 306 includes a cap portion 314 and tube receiving portion 316. Cap portion 314 includes a cap 318 and connector portion 320. Cap portion 314 can be threadably or snappably, removably connected to the port (not shown). Tube receiving portion 320 can include ridges 322 for receiving tubing 302 in a fluid tight friction fit.

Handle portion 312 can include tube receiving supports 324 for receiving tube 302. Detachable strap 326 can be provided for securing the tube 302 in place on the tube receiving supports 324. With reference to FIGS. 15A and B, the reservoir 301 can be removably received within a backpack 328. The backpack 328 can include a tube receiving aperture 312.

Various embodiments of systems, devices, and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the invention.

Persons of ordinary skill in the art will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features. The invention can comprise a combination of different individual features selected from different individual embodiments, as understood be persons of ordinary skill in the art. Further, elements described with respect to ne embodiment can be implemented in other embodiments even when not described in such embodiments unless otherwise noted. These combinations are proposed herein unless it is stated that a specific combination is not intended.

What is claimed is:

1. A urostomy collection system, comprising:
a collection vessel, the collection vessel comprising a body and a handle, wherein the body defines an internal cavity;
a cap removably couplable to an opening in a top face of the collection vessel;
a drainage tube;
a vessel connection point coupled to a first end of the drainage tube, wherein the vessel connection point is removably couplable to the cap; and
an ostomy connection point coupled to a second end of the drainage tube.

2. The urostomy collection system of claim 1, the collection vessel further comprising a base and a lid, the lid further comprising a handle and a transparent window in a top face of the lid, and wherein the base is coupled to a bottom face of the body of the collection vessel.

3. The urostomy collection system of claim 1, wherein the base comprises an elastomeric material.

4. The urostomy collection system of claim 1, wherein the internal capacity of the collection vessel is a gallon.

5. The urostomy collection system of claim 1, wherein the collection vessel comprises an opaque material.

6. The urostomy collection system of claim 1, wherein the ostomy connection point is removably couplable with a conventional urostomy port.

7. The urostomy collection system of claim 1, wherein the lid is removably coupled to the body of the collection vessel.

8. The urostomy collection system of claim 1, the collection system further comprising a plurality of tube stabilizers affixed to a top face of the handle and a tube sleeve.

9. The urostomy collection system of claim 1, wherein the urostomy collection system further comprises a transport bag.

10. The urostomy collection system of claim 1, wherein the collection vessel is removably inserted into the transport bag.

11. A method for expelling, containing, and disposing effluent from a urostomy port, the method comprising:
directing effluent from an ostomy port through a drainage tube, wherein the drainage tube is removably couplable to the ostomy port via an ostomy connector;
draining the effluent from the drainage tube into a collection vessel, wherein the drainage tube is removably coupled to a cap of the collection vessel via a vessel connector;
storing the effluent in an internal cavity of the collection vessel for a duration of time;
disengaging the drainage tube from the collection vessel by removing the vessel connector from the cap;
removing the cap from the collection vessel;
emptying the effluent from the collection vessel;
reattaching the cap to the collection vessel; and
reattaching the drainage tube to the collection vessel by reconnecting the vessel connector to the cap.

12. The method for expelling, containing, and disposing effluent from a urostomy port of claim 11, the collection vessel further comprising a body, a removably couplable lid, a transparent window on a top face of the lid, and an elastomeric base affixed to a bottom face of the body.

13. The method for expelling, containing, and disposing effluent from a urostomy port of claim 11, the method further comprising removing the removably couplable lid from the collection vessel such that a user can fully disinfect the internal cavity of the collection vessel.

14. The method for expelling, containing, and disposing effluent from a urostomy port of claim 11, the method further comprising storing the collection vessel in a transport bag.
